(19) United States
(12) Patent Application Publication
(10) Pub. No.: US 2013/0211215 A1
(43) Pub. Date: Aug. 15, 2013

(54) HYPEROSMOTIC PREPARATIONS COMPRISING 5-AMINOLEVULINIC ACID OR DERIVATIVE AS PHOTOSENSITIZING AGENT

(71) Applicants: Inger Ferner Heglund, Nesoya (NO); Aslak Godal, Oslo (NO); Jo Klaveness, Oslo (NO)

(72) Inventors: Inger Ferner Heglund, Nesoya (NO); Aslak Godal, Oslo (NO); Jo Klaveness, Oslo (NO)

(73) Assignee: Photocure ASA, Osio (NO)

(21) Appl. No.: 13/806,578

(22) PCT Filed: Jun. 23, 2011
(86) PCT No.: PCT/EP2011/060574

§ 371 Date: Apr. 17, 2013

(30) Foreign Application Priority Data
Jun. 23, 2010 (EP) 10251132.6

(51) Int. Cl. A61K 41/00; A61B 5/00

(52) U.S. Cl. CPC A61K 41/0061; A61B 5/0071; A61B 5/0084
USPC 600/317; 514/561; 604/500

(57) ABSTRACT

Provided herein are improved methods of photodynamic treatment and diagnosis of cancer and non-cancerous conditions in the gastrointestinal tract, e.g. in the colon, and in particular hyperosmotic enema preparations for use in such methods. The enema preparations comprise a photosensitizer which is 5-aminolevulinic acid (5-ALA) or a precursor or derivative thereof, e.g. a 5-ALA ester, in combination with at least one hyperosmotic agent. The methods and preparations herein described are particularly suitable for use in photodynamic methods of treating and/or diagnosing colorectal cancer.

The present invention relates to hyperosmotic preparations and their use in methods of photodynamic treatment and/or diagnosis of abnormalities, including cancer and non-cancerous conditions, in the gastrointestinal tract. In particular, it relates to hyperosmotic preparations for use in the early detection of colon cancer.

Photodynamic therapy (PDT) is a relatively new technique that has been used in the treatment of various cancers as well as other diseases. PDT involves the administration of photosensitizing agents followed by exposure to photoactivating light in order to activate the photosensitizing agents and convert them into cytotoxic form resulting in the destruction of cells and thus treatment of the disease. Several photosensitizing agents are known and described in the literature including 5-aminolevulinic acid (5-ALA) and certain derivatives thereof, e.g. 5-ALA esters.

Currently three pharmaceutical products comprising 5-ALA or an ester thereof are in clinical use for PDT and photodynamic diagnosis (PDD). These are Metvix® (Galderma, Switzerland), Hexvix® developed by Photocure ASA (Oslo, Norway) and Levulan Kerastick® developed by DUSA Pharmaceuticals (Canada). Metvix® is a dermal product for treatment of actinic keratosis and basal cell carcinoma which comprises methyl ALA ester in an emulsion (cream). Hexvix® is an aqueous solution which comprises hexyl ALA ester for instillation into the urine bladder for diagnosis of bladder cancer. Levulan Kerastick® is a 2-compartment formulation that is used to prepare a solution of 5-ALA immediately before application. This product can be used for the treatment of skin diseases.

An area of the body which is especially difficult to treat using PDT or PDD methods is the gastrointestinal tract, in particular the lower part of the g.i. tract such as the colon and rectum which may be associated with a number of serious and life-threatening diseases like colitis, colorectal cancer, Crohn's disease, irritable bowel disease and various local infections. Potentially the most serious of these is colorectal cancer.

Current diagnostic methods for colorectal cancer include monitoring of clinical symptoms like blood in the stools, lower abdominal pain, weight loss, coloscopy and X-ray based imaging methods. The prognosis of patients with colorectal cancer depends, as with most other cancer forms, on disease stage at the time of diagnosis and especially on whether the patient has developed distant metastasis. There are several therapeutic drugs in clinical use today for treating colorectal cancer, however, current drugs have their clinical limitations and there remains a medical need for further therapeutic regimes and alternative methods of early diagnosis.

Oral formulations comprising 5-ALA and derivatives thereof, such as solutions, suspensions, classical tablets and capsules containing aqueous formulations may have several disadvantages when used for the diagnosis and/or therapy of conditions in the lower part of the gastrointestinal system. These relate to shelf life stability of the pharmaceutical product, in vivo stability of the product during its passage through the whole gastrointestinal system, and systemic toxicity as a result of absorption of 5-ALA or derivatives thereof. Systemic absorption results in a reduction in clinical efficacy at the desired treatment site. Reduced efficacy is primarily a result of a non-homogenous and low concentration of 5-ALA or derivatives thereof reaching the lower part of the gastrointestinal system. In order for oral formulations to develop the desired clinical effects, it therefore becomes necessary for the amount of active ingredients to be increased. However, this can cause adverse reactions.

An alternative to oral formulations is the use of an enema in which a liquid containing 5-ALA or a derivative of 5-ALA is directly introduced into the rectum and colon; this has the advantage that the photosensitizing agent is directly administered to the desired target site without passing through the upper part of the gastrointestinal system.

A number of clinical studies have been carried out using enemas comprising 5-ALA and 5-ALA esters in the detection of certain abnormalities in the colon. For example, B. Mayinger et al. in Endoscopy 40: 106-109, 2008 describe a clinical study on detection of pre-malignant conditions in the colon by photodynamic diagnosis using enemas comprising 5-aminolevulinic acid hexyl ester (HAL) and fluorescence endoscopy as a means of detection. The enemas used in the study comprise 200 mg 5-aminolevulinic acid hexyl ester dissolved in 500 ml or 1000 ml sterile phosphate buffered saline. The authors show that the use of PDD detects 28% more polyps than when using white light endoscopic imaging. Similarly, E. Endlicher et al. in Gastrointestinal Endoscopy 60(3): 449-454, 2004 use a 5-ALA hexyl ester enema for the photodynamic detection of rectal adenoma or rectal cancer in patients. Messmann et al. in Gut 52: 1003-1007, 2003 use a 5-ALA enema for the photodynamic detection of low and high grade dysplasia in patients with ulcerative colitis.

Hyperosmotic enemas are known, such as Microlax® (McNeil) containing 3430 mM (3430 mOsm/l) sorbitol, and Klyx® (Ferring Legemidler AS) which contains 1370 mM (1370 mOsm/l) sorbitol. However, these enemas have not been proposed for use together with any photosensitizing agent.

The present inventors have now found through pharmacokinetic studies using 14C-labelled agents that the use of enema preparations can result in absorption of a significant amount of the active photosensitizing agent in the colon, particularly in the case of water soluble agents, such as hexyl ALA ester (HAL). Indeed, when carrying out studies involving the use of HAL enemas, the inventors frequently observed that the entire enema volume (e.g. 250 to 500 ml) was completely absorbed by the colon by the end of the instillation period (e.g. 30 to 60 mins); although not wishing to be bound by theory, it is considered likely that HAL is removed from the colon by this effective water uptake. Such high systemic uptake of photosensitizer may constitute a safety issue for the patient due to the high dosage of agent circulating within the bloodstream.

One potential solution to this problem is to reduce the amount of active agent which is administered; however, this may result in delivery of a sub-optimal dose and thus ineffective treatment or diagnosis. As an alternative to lowering the dose of active agent, the inventors have discovered that the problem of high systemic uptake of photosensitizer can be effectively addressed by reversing the normal osmotic gradient in the lower gastrointestinal tract (e.g. the colon) thereby achieving essentially a ‘steady-state’ with respect to water absorption from the lumen across the epithelial lining of the gut. This may be achieved using a hyperosmotic product. Such a product allows the administration of higher doses of the photosensitizer in cases where this may be desirable to obtain an optimal therapeutic or diagnostic result. Due to the low absorption of photosensitizer, this also results in an acceptable toxicity profile.

Provided herein are hyperosmotic products comprising a photosensitizing agent which is 5-ALA, a precursor or a derivative thereof, in a hyperosmotic formulation or solution.

Also provided herein are hyperosmotic preparations comprising a photosensitizing agent which is 5-ALA, a precursor or a derivative thereof, and at least one hyperosmotic agent.

The products herein described may contain a hyperosmotic agent, such as sorbitol, but this is not used at the high concentrations in known hyperosmotic enema agents. Instead this is used, for example, to reduce water uptake from the gastrointestinal tract. Hyperosmotic enemas, such as Microlax® (McNeil) containing 3430 mM (3430 mOsm/l) sorbitol, and Klyx® (Ferring Legemidler AS) containing 1370 mM (1370 mOsm/l) sorbitol are examples of known enema preparations. However, these enemas are not used together with any photosensitizing agent (e.g. 5-ALA or a 5-ALA ester) and are used for a completely different purpose; in these products the high sorbitol concentration is intended to draw water into the colon and “dissolve” the faeces thereby relieving constipation.

The hyperosmotic products and preparations herein described may further comprise at least one pharmaceutically acceptable carrier or excipient. However, in certain cases, the hyperosmotic agent itself may act as a suitable carrier or excipient such that no additional carrier need be present.

In one embodiment, the hyperosmotic preparation is a liquid preparation which comprises a liquid carrier, preferably an aqueous carrier. Suitable carriers include, for example, an aqueous buffer or water.

In another aspect, provided herein are hyperosmotic preparations comprising a photosensitizing agent which is 5-ALA, a precursor or a derivative thereof, and at least one hyperosmotic agent, for use in medicine or for use as a medicament, particularly for use in the photodynamic treatment or diagnosis of cancer or a non-cancerous condition in the lower part of the gastrointestinal tract.

In a further aspect, provided herein is a method of photodynamic treatment or diagnosis of cancer or a non-cancerous condition in a patient, said method comprising the steps of:
(i) administering to said patient an effective amount of a hyperosmotic preparation comprising a photosensitizing agent which is 5-ALA, a precursor or a derivative thereof, and at least one hyperosmotic agent;
(ii) optionally waiting for a time period for the photosensitizing agent to achieve an effective tissue concentration at the desired site; and
(iii) photoactivating the photosensitizing agent.

In another aspect, provided herein is the use of any of the preparations herein described in the photodynamic treatment or diagnosis of cancer in the lower part of the gastrointestinal tract, especially colorectal cancer.

In a preferred embodiment, the hyperosmotic preparations herein described will be provided in the form of an enema.

The diagnostic methods described herein may also be performed during surgery in which the preparation is given to the patient prior to surgery and surgery is then performed under light which causes the photosensitizer to fluoresce. The fact that the lesion or disease fluoresce aids the surgeon in defining the “surgical border” and thereby enables a more selective resection of the diseased area, e.g. a tumour. Also provided herein is thus the use of the preparations herein described in methods of surgery.

The therapeutic and diagnostic methods herein described may also be used in the form of a combined therapy. For example, a course of PDT performed in relation to a cancerous or non-cancerous condition using any of the methods herein described may be followed by a PDD method, e.g. to determine the extent to which PDT has been effective and/or to detect any re-occurrence of the condition. Also, a course of PDD performed in relation to a cancerous or non-cancerous condition using any of the methods herein described may be followed by a PDT method, e.g. to treat cancerous or non-cancerous conditions which have been detected by PDD.

In a further aspect, provided herein is a hyperosmotic preparation as herein described for use in a method which comprises the steps of:
(i) conducting photodynamic treatment of cancer or a non-cancerous condition in the lower part of the gastrointestinal system, e.g. the colon or rectum, of a patient; and subsequently
(ii) conducting photodynamic diagnosis on said patient.

At least one of steps (i) and (ii) is performed following administration to the patient of a preparation as provided herein. Preferably, steps (i) and (ii) will both be performed following administration of such a preparation.

Also provided herein is a hyperosmotic preparation as herein described for use in a method which comprises the steps of:
(iii) conducting photodynamic diagnosis of cancer or a non-cancerous condition in the lower part of the gastrointestinal system, e.g. the colon or rectum, of a patient; and subsequently
(iv) conducting photodynamic treatment on said patient.

At least one of steps (iii) and (iv) is performed following administration to said patient of a preparation as provided herein. Preferably, steps (iii) and (iv) will both be performed following administration of such a preparation.

The term “hyperosmotic” is used herein to describe a preparation (e.g. a solution) having an osmotic pressure greater than that of a physiologic salt solution. Preparations having an osmolarity greater than about 300 mOsm per litre (at ambient temperature) are generally considered “hyperosmotic”. A “hyperosmotic agent” should be construed accordingly and is intended to encompass any substance which is capable of increasing the hyperosmoticity of a preparation. Provided herein is the use of both penetrating and non-penetrating solutes as agents to obtain hyperosmotic solutions.

An osmometer may be used to determine the osmolarity of a solution. There are several different techniques employed in osmometry; vapour pressure depression osmometers determine the concentration of osmotically active particles that reduce the vapour pressure of a solution; membrane osmometers measure the osmotic pressure of a solution separated from pure solvent by a semi-permeable membrane; and freezing point depression osmometers are used to determine the osmotic strength of a solution (since osmotically active compounds depress the freezing point of a solution). Osmolarity may be measured using a vapour pressure osmometer such as that supplied by ELITech Group (Vapro 5600 vapour pressure osmometer). Where reference is made herein to specific values for osmolarity, these values may be determined using such apparatus operated under standard temperature and pressure conditions.

Osmolarity is a measure of the concentration of solute in a solution and is defined as the number of osmoles (Osm) of solute per litre of solution (i.e. Osm/l). The osmole (Osm) is a unit of measurement which defines the number of moles of a substance that contributes to the osmotic pressure of the solution.

The osmolarity of small molecules can normally be calculated from their concentration in solution. For example, in the case of simple salts such as NaCl: 0.9% NaCl=150 mM. NaCl is fully dissociated in water and both Na⁺ and Cl⁻ contribute to the osmolarity (i.e. each mole of NaCl becomes two osmoles in solution, one mole of Na⁺ and one mole of Cl⁻). The osmolarity is therefore 150 mM×2=300 mOsmol/l. For sorbitol, which does not dissociate in water, the osmolarity equals the concentration (300 mM=300 mOsmol/l). In the case of complex salts such as sodium phosphate the concentration of each of the phosphate species present in solution depends on the solution pH.

For the photosensitizing agents herein described, such as ALA hexylester HCl, the active will dissociate in solution into Cl⁻ and ALA hexylester with an extra proton on its amino group (note this will not dissociate further since the pH of the solution will be around 5 for stability reasons). The osmolarity will therefore be twice the active agent concentration (e.g. 20 mM ALA hexylester=40 mOsmol/l).

The osmolarity of large molecules (e.g. polymers like PEG) can not be calculated directly from their concentration, but information can be found in suitable handbooks of Physics and Chemistry or from publications. Otherwise, the osmolarity may be determined using an instrument such as that described herein.

The osmolarity of a solution is dependent on the presence of other solutes in the solution. Any reference herein to osmolarity is intended to refer to the total osmolarity of the final solution. Where it is desired to achieve a solution having a particular osmolarity, it will readily be appreciated that the exact concentration of the hyperosmotic agent may have to be adjusted depending on the concentration and properties of the other components in the solution (e.g. salt and buffer ions, the active photosensitizing agent, any other excipients or carriers, any other actives, etc.).

Following administration of a hyperosmotic preparation as herein described, water will be drawn into the colon by osmosis. Too high an influx of water should, however, be avoided since this may result in an increased volume within the colon (which can be unpleasant for the patient) and an unacceptable level of dilution of the active photosensitizing agent. Preferred for use in the methods described herein are hyperosmotic solutions which are effective in preventing unacceptable levels of water uptake leading to systemic uptake of the water-soluble photosensitizing agent from the gastrointestinal tract whilst at the same time avoiding too high an influx of water into the colon (and potentially dehydration). Appropriate levels of hyperosmoticity may readily be determined by those skilled in the art. Hyperosmotic solutions having an osmolarity in excess of about 300 mOsm/l, e.g. about 310 mOsm/l, or in the range 320 to 900 mOsm/l, or in the range 350 to 650 mOsm/l, or in the range of 350 to 500 mOsm/l, are generally useful in the methods described herein (values measured at ambient temperature, i.e. 18 to 25° C.).

As used herein, the terms “cancer” and “cancerous” are used in connection with conditions where malignant cells are present. Pre-malignant conditions are thus not encompassed by these terms.

The term “non-cancerous” may include pre-malignant conditions. However, preferred non-cancerous conditions for treatment in accordance with the methods and formulations described herein are those which are not pre-malignant. Examples of non-cancerous conditions are inflammatory diseases such as inflammatory bowel diseases, particularly Crohn's disease or ulcerative colitis, and infectious diseases such as infections caused by bacteria (e.g. clostridium difficile which may lead to pseudomembraneous colitis) or parasites (e.g. trichuriasis).

As used herein the term “treatment” or “therapy” encompasses curative as well as prophylactic treatment or therapy.

The preparations herein described are generally provided in a form suitable for administration as an enema. For example, these may be provided in disposable bags or bottles connected to tubing. Hyperosmotic enema preparations form a particularly preferred aspect of the formulations and methods disclosed herein.

Hyperosmotic preparations as herein described comprise one or more hyperosmotic agents which serve to increase the hyperosmoticity of the solution. Such agents are well known and used in the art and include, for example, conventional osmotic laxatives which function by drawing water into the gut through their osmotic action.

Examples of suitable hyperosmotic agents for use in the methods and formulations disclosed herein include salts, sugars, sugar alcohols, glycerol and polyols. Hyperosmotic agents which are not themselves taken up systemically from the colon are particularly preferred and include, in particular, sugars, sugar alcohols, glycerol and polyols (e.g. PEG).

Suitable salts include substances known and used as saline laxatives, in particular those which comprise ions which are poorly absorbed from the gut. Those based on sodium phosphate, magnesium citrate and other magnesium salts are particularly preferred. Specific examples of suitable salts include magnesium sulphate, magnesium hydroxide, magnesium citrate, magnesium chloride and sodium phosphate. Other salts which may be used include sodium sulphate, potassium sodium tartarate, sodium chloride, sodium bicarbonate, potassium chloride, calcium chloride and calcium gluconate, although these are generally less preferred. Combinations of any of these salts may also be used.

Phosphate salt preparations suitable for use in the methods and formulations disclosed herein include those containing a combination of monobasic sodium phosphate and dibasic sodium phosphate. One such preparation is that sold under the tradename Fleet Phospho-Soda® or Phosphoral® (Laboratoires Casen-Fleet S.L.U., Spain).

Sugar alcohols may also be used as hyperosmotic agents in the methods and formulations disclosed herein. Those which are poorly absorbed (i.e. indigestible) are particularly useful and include sorbitol, mannitol, lactitol and xylitol. Particularly preferred are sorbitol, mannitol and xylitol. Combinations of any of the sugar alcohols may also be used.

Amongst the sugars which may be used as hyperosmotic agents are both natural and synthetic sugars including lactulose, fructose, galactose and lactose, or any combinations thereof. One example of such a product is Duphalac® (Solvay Healthcare Limited, UK) which comprises lactulose, fructose, galactose and lactose.

Polyols may also be used as hyperosmotic agents. Polyether polyols are particularly preferred and include polyethylene glycols (PEGs) and polyethylenepolypropylene glycols (PPGs). Examples of polyether polyols are polyethylene glycol, polypropylene glycol, polyethylene-polypropylene glycol block copolymer and random polymers and polybutylene polyols.

In one embodiment the hyperosmotic agent may be a polyethylene glycol. Any food or pharmaceutical grade PEG polymer may be employed. Those which have a relatively high molecular weight and which are thus solid at room temperature are generally preferred. These may be soluble in water or, alternatively, miscible with water at room temperature to provide an aqueous suspension of a PEG. PEG polymer having an average molecular weight in the range between 1,000 and 25,000 daltons, preferably between about 2,000 and about 10,000 daltons, for example between about 3,000 and about 4,000 daltons may be used. In a preferred embodiment the osmotic agent is a polyethylene glycol having an average molecular weight of about 3,350 daltons, i.e. PEG (3350). PEG (4000) may also be used. Such agents are commercially available, e.g. from the Dow Chemical Company, USA.

Other PEG containing products which are commercially available are those comprising PEG in combination with an isotonic mixture of electrolytes. These include, in particular, Endofalk® (Dr. Falk Pharma GmbH, Germany), Laxabon® (Recipharm Höganäs AB, Sweden), Movicol® (Norgine, Norway) and Molaxole® (Meda Pharmaceuticals, UK). In each of these products the active osmotic agent is macrogol 3350 (PEG 3350).

Another polyether polyol which may be used is polyethylenepolypropylene glycol (PPG). PPGs are also known under the name pluronic and are available in a range of molecular weights. Suitable products include Pluronic F68 and Poloxamer 188.

Combinations of any of the hyperosmotic agents herein described herein may also be used in the methods and formulations disclosed herein. In particular, any of the agents which on dissolution in a suitable carrier provide non-penetrating solutes (i.e. the sugars, sugar alcohols, glycerol and polyols) may be used in combination with any of the agents which, in solution, provide penetrating solutes (i.e. any of the salts which are herein described, especially NaCl).

The concentration of the hyperosmotic agent required to obtain the desired osmotic pressure may readily be determined by those skilled in the art and will vary depending on the nature of the agent selected. An optimal concentration of the agent is one which results in little in or out flow of water across the lining of the gastrointestinal tract. As will be readily appreciated, it may be important to limit the concentration of certain hyperosmotic agents, especially salts, to avoid adverse effects. For example, an increase in sodium chloride concentration in the blood can lead to potential systemic side effects such as an increase in blood pressure. Similarly, phosphate salts should be used in relatively low concentrations since a high concentration of phosphate ions in the blood can have a toxic effect due to binding with calcium ions.

Generally, if dissolved in physiological saline, the concentration of the hyperosmotic agent may range from about 10 mM to 1 M. In one embodiment, the formulation may contain from about 20 to 900 mM of the hyperosmotic agent. In another embodiment, the amount of the hyperosmotic agent may range from about 30 to about 500 mM, or from about 50 to about 500 mM. However, if dissolved in water, the corresponding ranges may be from about 310 mM to 1 M, or from about 320 to 900 mM, or from about 350 to about 600 mM, or from about 350 to about 500 mM.

The term “precursors” as used herein refers to precursors for 5-ALA which are converted metabolically to it and are thus essentially equivalent thereto. Thus the term “precursor” covers biological precursors for protoporphyrin in the metabolic pathway for haem biosynthesis. The term “derivatives” includes pharmaceutically acceptable salts and chemically modified agents, for example esters such as 5-ALA esters.

The use of 5-ALA and derivatives thereof, e.g. 5-ALA esters in PDT and PDD is well known in the scientific and patent literature (see, for example, WO 2006/051269, WO 2005/092838, WO 03/011265, WO 02/09690, WO 02/10120, WO 2003/041673 and U.S. Pat. No. 6,034,267, the contents of which are incorporated herein by reference). All such derivatives of 5-ALA and their pharmaceutically acceptable salts are suitable for use in the methods and formulations disclosed herein.

The synthesis of 5-ALA is known in the art. Further, 5-ALA and pharmaceutically acceptable salts thereof are commercially available, for instance from Sigma Aldrich.

The 5-ALA derivatives which may be used in the methods and formulations disclosed herein may be any derivative of 5-ALA capable of forming protoporphyrins, e.g. protoporphyrin IX (PpIX) or any other photosensitizer, e.g. a PpIX derivative in vivo. Such derivatives may be a precursor of PpIX or of a PpIX derivative, e.g. a PpIX ester, in the biosynthetic pathway for haem and which are therefore capable of inducing an accumulation of PpIX in vivo at the site of the administration. Suitable precursors of PpIX or PpIX derivatives include 5-ALA prodrugs which might be able to form 5-ALA in vivo as an intermediate in the biosynthesis of PpIX or which may be converted, e.g. enzymatically converted, to porphyrins without forming 5-ALA as an intermediate. 5-ALA esters and pharmaceutically acceptable salts thereof, are among the preferred photosensitizers for use in the methods and formulations disclosed herein.

Esters of 5-aminolevulinic acid and N-substituted derivatives thereof are preferred photosensitizers for use in the methods and formulations disclosed herein. Those compounds in which the 5-amino group is unsubstituted, i.e. the ALA esters, are preferred. Such compounds are generally known and described in the literature (see, for example, WO 96/28412 and WO 02/10120 to Photocure ASA, the contents of which are incorporated herein by reference).

Esters of 5-aminolevulinic acid with substituted or unsubstituted alkanols, i.e. alkyl esters and substituted alkyl esters, are preferred photosensitizers for use in the methods and formulations disclosed herein. Examples of such compounds include those of formula I:

R²₂N—CH₂COCH₂—CH₂CO—OR¹ (I)

wherein

R¹ represents a substituted or unsubstituted alkyl group, preferably an unsubstituted alkyl group; and

R² each independently represents a hydrogen atom or a group R¹, preferably a hydrogen atom.

As used herein, the term “alkyl”, unless stated otherwise, includes any long or short chain, cyclic, straight-chained or branched, saturated or unsaturated aliphatic hydrocarbon group. The unsaturated alkyl groups may be mono- or polyunsaturated and include both alkenyl and alkynyl groups. Unless stated otherwise, such alkyl groups may contain up to 40 carbon atoms. However, alkyl groups containing up to 30 carbon atoms, preferably up to 10, particularly preferably up to 8, especially preferably up to 6 carbon atoms are preferred.

In compounds of formula I, the R¹ groups are substituted or unsubstituted alkyl groups. If R¹ is a substituted alkyl group, one or more substituents are either attached to the alkyl group and/or interrupt the alkyl group. Suitable substituents that are attached to the alkyl group are those selected from: hydroxy, alkoxy, acyloxy, alkoxycarbonyloxy, amino, aryl, nitro, oxo, fluoro, —SR³, —NR³₂ and —PR³₂ wherein R³ is a hydrogen atom or a C₁₋₆ alkyl group. Suitable substituents that interrupt the alkyl group are those selected from: —O—, —NR³—, —S— or —PR³.

If R¹ is a substituted alkyl group, one or more aryl substituents, i.e. aryl groups, preferably one aryl group, are preferred.

As used herein, the term “aryl group” denotes an aromatic group which may or may not contain heteroatoms like nitrogen, oxygen or sulphur. Aryl groups which do not contain heteroatoms are preferred. Preferred aryl groups comprise up to 20 carbon atoms, more preferably up to 12 carbon atoms, for example, 10 or 6 carbon atoms. Examples of aryl groups are phenyl and naphthyl, especially phenyl. Further, the aryl group may optionally be substituted by one or more, more preferably one or two, substituents. The aryl group may be substituted at the meta or para position, most preferably the para position. Suitable substituents include haloalkyl, e.g. trifluoromethyl, alkoxy, e.g. alkoxy groups containing 1 to 6 carbon atoms, halo (e.g. iodo, bromo, chloro or fluoro, preferably chloro and fluoro), nitro and C₁₋₆ alkyl, preferably C₁₋₄ alkyl. For example, C₁₋₆ alkyl groups include methyl, isopropyl and t-butyl, particularly methyl. Exemplary aryl substituents are chloro and nitro. However, the aryl group may be unsubstituted.

R¹ groups may include, for example, benzyl, 4-isopropylbenzyl, 4-methylbenzyl, 2-methylbenzyl, 3-methylbenzyl, 4-[t-butyl]benzyl, 4-[trifluoromethyl]benzyl, 4-methoxybenzyl, 3,4-[di-chloro]benzyl, 4-chlorobenzyl, 4-fluorobenzyl, 2-fluorobenzyl, 3-fluorobenzyl, 2,3,4,5,6-pentafluorobenzyl, 3-nitrobenzyl, 4-nitrobenzyl, 2-phenylethyl, 4-phenylbutyl, 3-pyridinyl-methyl, 4-diphenyl-methyl and benzyl-5-[(1-acetyloxyethoxy)-carbonyl]. Preferred R¹ groups are benzyl, 4-isopropylbenzyl, 4-methylbenzyl 4-nitrobenzyl and 4-chlorobenzyl, e.g. benzyl.

If R¹ is a substituted alkyl group, one or more —O— substituents are preferred. Such groups may be straight-chained C₄₋₁₂ alkyl groups which are substituted by one or more —O— groups, preferably by one to five —O— groups. The —O— groups may be present in the alkyl group in an alternating order, i.e. resulting in short chain polyethylene glycol substituents. Examples of such groups include 3,6-dioxa-1-octyl and 3,6,9-trioxa-1-decyl.

If R¹ is an unsubstituted alkyl group, R¹ groups that are saturated straight-chained or branched alkyl groups are preferred. If R¹ is a saturated straight-chained alkyl group, C₁₋₁₀ straight-chained alkyl group are preferred. Representative examples of suitable straight-chained alkyl groups include methyl, ethyl, n-propyl, n-butyl, n-pentyl, n-hexyl and n-octyl. Examples include C₁₋₆ straight-chained alkyl groups. Most particularly preferred are C₃-C₆ straight-chained alkyl groups, e.g. n-hexyl. If R¹ is a saturated branched alkyl group, such branched alkyl groups preferably consists of a stem of 4 to 8, preferably 5 to 8 straight-chained carbon atoms which is branched by one or more C₁₋₆ alkyl groups, preferably C₁₋₂ alkyl groups. Examples of such saturated branched alkyl groups include 2-methylpentyl, 4-methylpentyl, 1-ethylbutyl and 3,3-dimethyl-1-butyl.

In compounds of formula I, each R² independently represents a hydrogen atom or a group R¹. Preferred for use in the methods and formulations disclosed herein are those compounds of formula I in which at least one R² represents a hydrogen atom. In especially preferred compounds each R² represents a hydrogen atom.

Preferred photosensitizers to be used in the preparations described herein are compounds of formula I and pharmaceutically acceptable salts thereof, wherein R¹ is hexyl, more preferably n-hexyl and both R² represent hydrogen, i.e. 5-ALA hexyl ester and pharmaceutically acceptable salts thereof, preferably the HCl salt or sulfonic acid or sulfonic acid derivative salts. The most preferred photosensitizer is 5-ALA hexyl ester in the form of its HCl salt.

5-ALA esters and pharmaceutically acceptable salts for use in the methods and formulations disclosed herein may be prepared by any conventional procedure available in the art, e.g. as described in WO 96/28412, WO 02/10120 and WO 2003/041673. For example, esters of 5-ALA may be prepared by reaction of 5-ALA with the appropriate alcohol in the presence of a catalyst, e.g. an acid or a base. Alternatively compounds for use in the methods and formulations disclosed herein may be available commercially, e.g. from Photocure ASA, Norway.

The 5-ALA esters for use as described herein may be in the form of a free amine, e.g. —NH₂, —NHR² or —NR²R², or preferably in the form of a physiologically acceptable salt. Such salts preferably are acid addition salts with physiologically acceptable organic or inorganic acids. Suitable acids include, for example, hydrochloric, nitric, hydrobromic, phosphoric, sulphuric, sulphonic and sulphonic acid derivatives. Particularly preferred salts are acid addition salts with sulphonic acid or sulphonic acid derivatives as described in WO 2005/092838 to Photocure ASA, the entire contents of which are incorporated herein by reference. Procedures for salt formation are well known in the art.

The preparations described herein may further comprise at least one liquid pharmaceutically acceptable carrier and optionally various excipients. The liquid may be water or a physiologically acceptable solvent or a mixture of water and one or more physiologically acceptable solvents. Such solvents include, for example, glycerol, ethylene glycol, propylene glycol, polyethylene glycol and polypropylene glycol. A particularly preferred liquid carrier is water. Aqueous hyperosmotic solutions are thus especially preferred.

In another embodiment, oils may be used as a solvent, e.g. natural and/or synthetic oils that are commonly used in pharmaceutical preparations. Examples of suitable natural oils are almond oil, olive oil, sunflower oil, soybean oil, palm kernel oil, corn oil, safflower oil, peanut oil, and coconut oil. Examples of suitable synthetic oils are hydrogenated or partially hydrogenated soybean oil, rapeseed oil, sunflower oil, coconut oil and fractions thereof or synthetic medium-chain triglycerides (MCT). Oils may be used in combination with an aqueous carrier, e.g. in combination with water or an aqueous buffer. If necessary, an emulsifier may be added. If oils are used, it is preferred to use a lipophilic salt of 5-ALA or a lipophilic salt and/or ester of 5-ALA, e.g. a mesylate or tosylate salt of 5-ALA or such a salt of a 5-ALA ester comprising an alkyl residue of 2-10 carbon atoms, such as hexyl ALA ester or benzyl ALA ester.

Further pharmaceutical excipients and carriers that may be used in the pharmaceutical products herein described are listed in various handbooks (e.g. D. E. Bugay and W. P. Findlay (Eds) Pharmaceutical excipients (Marcel Dekker, New York, 1999), E-M Hoepfner, A. Reng and P. C. Schmidt (Eds) Fiedler Encyclopedia of Excipients for Pharmaceuticals, Cosmetics and Related Areas (Edition Cantor, Munich, 2002) and H. P. Fielder (Ed) Lexikon der Hilfsstoffe für Pharmazie, Kosmetik and angrenzende Gebiete (Edition Cantor Aulendorf, 1989)).

Other known excipients such as buffers, preservatives, pH adjusters, etc. may also be included in the formulations herein described. These may contain a buffer system (e.g. a phosphate buffer) which serves to maintain the formulation at a pH of about 6 to 7.5, especially about 6.

In one aspect there is provided a formulation which comprises the photosensitizing agent (e.g. a 5-ALA ester), optionally a buffer system and/or NaCl, a hyperosmotic agent, and water.

The formulations herein described may also contain one or more agents selected from the following:
a) one or more viscosity enhancing agents;
b) one or more mucoadhesive agents; and
c) one or more chelating agents.

The term “one or more of the following” means that the preparations described herein may comprise at least one compound of the group of compounds a) to c), e.g. either a) or b) or c). Alternatively, the preparation may comprise more than one compound of the group of compounds a) to c), e.g. one or more viscosity enhancers a) and one or more chelating agents c).

The preparations may take any form which is suitable for administration, e.g. oral or intra-colonic administration, and which may include solution, suspension, sol and gel forms. The enemas herein described may take the form of a liquid (e.g. a solution or a suspension) or foam. Compositions of foam enemas are generally described in the prior art, see for example U.S. Pat. No. 6,432,967. Thus the carrier vehicle may also comprise an effective amount of a foaming agent such as n-butane, propane or iso-butane. Such formulations can be delivered from a pressurised container so that this is delivered to the colon as a foam which inhibits release from the target site.

For oral administration the preparations may, for example, comprise a solution in which the photosensitizing agent is dissolved or dispersed. These may be prepared at the point-of-use by dissolving or dispersing the photosensitizing agent in a physiologically acceptable solvent (e.g. water). Alternatively, these may be provided in ready-to-use form.

The photosensitizers herein described may be used for the manufacture of a hyperosmotic preparation in any manner. The desired concentration of photosensitizer in the preparations described herein will vary depending on several factors including the nature of the compound, the nature and form of the product in which this is presented, the nature of the condition (e.g. cancer) to be treated or diagnosed and the subject to be treated. Generally, however, the concentration of photosensitizer (e.g. hexyl ALA ester) is, for example, in the range 0.001 to 10 mmol per litre, from 0.01 to 5 mmol per litre, or from 0.05 to 4 mmol per litre. The photosensitizer may be used, for example, in a concentration of from 0.05 to 4 mmol per litre, e.g. less than 2.5 mmol per litre.

Alternatively, the photosensitizer (e.g. methyl ALA ester) may be used at a concentration in the range from 0.1 to 1000 mmol per litre, from 1 to 500 mmol per litre, or from 5 to 400 mmol per litre. The photosensitizer may be used, for example, in a concentration of from 5 to 400 mmol per litre, e.g. less than 250 mmol per litre.

The preparations herein described provide an essentially homogeneous filling of the entire colon following administration and optionally any movement of the patient. Further homogeneous filling of the colon may be achieved by using, for example, a) one or more a viscosity enhancing agents. The one or more viscosity enhancing agents can be any viscosity enhancing agent used in pharmaceutical formulations. Viscosity enhancing agents to be used in a preparation as herein described include, for example, gelatine, tragacanth gums, xanthan gums, pectin, polysaccharides and cellulose derivatives like carboxymethyl cellulose, methyl cellulose, hydroxypropyl cellulose, etc.

One aspect presented herein relates to enema preparations that change viscosity over time, for example, the viscosity is low during administration but increases after the enema is instilled into the area of interest. This can be achieved by administration of preparations comprising one or more viscosity agents which comprise swellable compounds, for example, polysaccharides, where the swellable compounds are not fully swollen before administration of the preparation. Alternatively, one or more viscosity agents may be used which increase the viscosity of the liquid when warmed up from around room temperature to body temperature. Several such viscosity agents are generally known in the art of galenic formulations.

The preparations described herein may comprise b) one or more mucoadhesive agents. Mucoadhesive agents help to improve adhesion to the colon wall and thus achieve uniform coating of the target site. As used herein, “mucoadhesive agent” refers, for example, to any agent which exhibits an affinity for a mucosa surface, e.g. which adheres to that surface through the formation of bonds which are generally non-covalent in nature, whether binding occurs through interaction with the mucous or the underlying cells. The mucoadhesive agent can be any mucoadhesive agent used in pharmaceutical formulations. Mucoadhesive agents to be used in the current formulations include those described in WO 02/09690, the entire contents of which are incorporated herein by reference.

Mucoadhesive agents which may be used in the preparations herein described may be natural or synthetic, polyanionic, polycationic or neutral, water-soluble or water-insoluble, but are preferably large, more preferably having a molecular weight of 500 to 3000 kDa, e.g. 1000 to 2000 kDa, water-insoluble cross-linked, e.g. containing 0.05 to 2%, e.g. 0.75 to 1.5% cross-linker by weight of the total polymer, prior to any hydration, water-swellable polymers capable of forming hydrogen bonds. Mucoadhesives may have a mucoadhesive force greater than 100, greater than 120, or greater than 150, as assessed according to the method of Smart et al., 1984, J. Pharm. Pharmacol., 36, p 295-299, expressed as a percent relative to a standard in vitro.

Appropriate mucoadhesive agents include, for example, poly(carboxylic acid-containing) based polymers, such as poly (acrylic, maleic, itaconic, citraconic, hydroxyethyl methacrylic or methacrylic) acid which have strong hydrogen-bonding groups, or derivatives thereof such as salts and esters. Alternatively, cellulose derivatives may be used such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof. Other naturally occurring or synthetic polymers may also be used such as gums, e.g. xanthan gum, guar gum, locust bean gum, tragacanth gums, karaya gum, ghatti gum, cholla gum, psillium seed gum and gum arabic; clays such as manomorillonite clays, e.g. Veegun, attapulgite clay; polysaccharides such as dextran, pectin, amylopectin, agar, mannan or polygalactonic acid or starches such as hydroxypropyl starch or carboxymethyl starch; lipophilic formulations containing polysaccharides, e.g. Orabase (Bristol Myers Squibb); carbohydrates such as polysubstituted with groups such as sulphate, phosphate, sulphonate or phosphonate, e.g. sucrose octasulphate; polypeptides such as casein, gluten, gelatin, fibrin glue; chitosan, e.g. lactate or glutamate or carboxymethyl chitin; glycosaminoglycans such as hyaluronic acid; metals or water soluble salts of alginic acid such as sodium alginate or magnesium alginate; schleroglucan; adhesives containing bismuth oxide or aluminium oxide; atherocollagen; polyvinyl polymers such as polyvinyl alcohols, polyvinylmethyl ethers, polyvinylpyrrolidone, polycarboxylated vinyl polymers such as polyacrylic acid as mentioned above; polysiloxanes; polyethers; polyethylene oxides and glycols; polyalkoxys and polyacrylamides and derivatives and salts thereof.

The above described polymeric mucoadhesive agent may also be cross-linked and may be in the form of copolymers. Poly(acrylic acid) polymers or copolymers, e.g. with di- or poly functional allyl ethers or acrylates may be used to make the polymer insoluble, which have preferably been cross-linked, e.g. using a polyalkenyl polyether, are employed which have a high molecular weight and are thixotropic. Appropriate mucoadhesive agents having this form are available commercially (e.g. from Goodrich) as polycarbophil, e.g. Noveon AA-1, Carbomer (Carbopol), e.g. Carbopol EX165, EX214, 434, 910, 934, 934P, 940, 941, 951, 974P and 1342.

Some of the preferred mucoadhesive agents for use in the preparations described herein include, for example, polyacrylic hydrogels, chitosan, polyvinyl alcohol, hydroxypropyl cellulose, hydroxypropyl methyl cellulose, sodium alginate, scleroglucan, xanthan gum, pectin, orabase and polygalactonic acid.

Some of the one or more compounds a) and b) impact on and prolong the release of the active photosensitizing agent. Such components are well known in the art and may include, for example, guar gum or other gums. The desired content of such components, e.g. gums, in the formulation can readily be determined by those skilled in the art and may, for example, be in the range 1 to 10 weight %.

The preparations described herein may comprise c) one or more chelating agents which have a beneficial effect in enhancing the accumulation of protoporphyrin (Pp) since the chelation of iron by the chelating agent prevents its incorporation into Pp to form haem by the action of the enzyme ferrochelatase, thereby leading to a build up of Pp. The photosensitizing effect is therefore enhanced. Enema preparations which include one or more chelating agents are thus particularly preferred since their use shortens the time of the enema procedure: less photosensitizer needs to be taken up into the tissue in one time unit to achieve a similar fluorescence compared to enemas without chelating agents. Alternatively, less amount of photosensitizer may be used in the enema preparation.

Suitable chelating agents include, for example, aminopolycarboxylic acids, such as any of the chelants described in the literature for metal detoxification or for the chelation of paramagnetic metal ions in magnetic resonance imaging contrast agents. Particular mention may be made of EDTA, CDTA (cyclohexane triamine tetraacetic acid), DTPA and DOTA and well known derivatives and analogues thereof. EDTA and DTPA are particularly preferred. To achieve the iron-chelating effect, desferrioxamine and other siderophores may also be used, e.g. in conjunction with aminopolycarboxylic acid chelating agents such as EDTA.

Where present, the one or more chelating agents may be used at a concentration of 0.05 to 20%, e.g. 0.1 to 10% by weight based on the preparation in which it is present.

In order to prepare the formulations herein described, at least one hyperosmotic agent may be dissolved or dispersed in a pharmaceutically acceptable carrier or excipient, for example water or physiological saline, to which the active photosensitizing agent may then be added. Where the hyperosmotic agent itself also functions as a carrier, the formulations may be produced by simple admixture of this with the photosensitizing agent.

Prior to carrying out the therapeutic and diagnostic methods herein described it is preferred that the lower part of the gastrointestinal tract, e.g. the colon and rectum, should be evacuated, i.e. cleansed. This may be achieved in several ways known in the art, for example using an enema procedure such as the use of an isotonic saline enema or the administration of laxative medications which may be taken orally. Products for cleansing include bisacodyl suppositories like Laxbene® (Merckle GmbH, Germany), oral formulations like Delcoprep® (DeltaSelect, Germany) and Endofalk® (DR. Falk GmbH, Germany), enemas comprising bisacodyl like Toilax® (Orion, Finland), rectal solutions containing sodium dioctylsulphosuccinate like Klyx (Ferring, Sweden) and enemas comprising sodium lauryl sulphate like Microlax® (McNeil, Sweden). The patient may also be required to fast, e.g. for a period of up to 12 hours prior to treatment.

In a further aspect, provided herein is a method of photodynamic treatment or diagnosis of cancer or a non-cancerous condition in a patient, said method comprising the steps of:
(i) evacuating the lower part of the gastrointestinal system of said patient;
(ii) optionally insufflating the lower part of the gastrointestinal system, e.g. with air or a gas;
(iii) administering to said patient a hyperosmotic preparation as herein described;
(iv) optionally waiting for a time period necessary for the photosensitizing agent to achieve an effective tissue concentration at the desired site;
(v) optionally insufflating the lower part of the gastrointestinal system, e.g. with air or a gas; and
(vi) photoactivating the photosensitizing agent.

In certain embodiments, step (ii) may be omitted. Preferably, steps (ii) and (v) may be omitted. In an alternative embodiment, the method may further comprise the step of evacuating the lower part of the gastrointestinal system of the patient after the hyperosmotic enema preparation has been administered and prior to photoactivation of the photosensitizing agent.

In one embodiment, the method of photodynamic treatment or diagnosis of cancer or a non-cancerous condition in a patient comprises:
(i) evacuating the lower part of the gastrointestinal system of the patient;
(ii) administering to the patient a hyperosmotic preparation as herein described; and
(iii) photoactivating the photosensitizing agent.

In another embodiment, the method of photodynamic treatment or diagnosis of cancer or a non-cancerous condition in a patient comprises:
(i) evacuating the lower part of the gastrointestinal system of the patient;
(ii) insufflating the lower part of the gastrointestinal system;
(iii) administering to the patient a hyperosmotic preparation as herein described; and
(iv) photoactivating the photosensitizing agent.

In a further aspect, provided herein is a method of photodynamic treatment or diagnosis of cancer or a non-cancerous condition in a patient, said method comprising:
(i) evacuating the lower part of the gastrointestinal system of the patient;
(ii) administering to the patient a hyperosmotic preparation as herein described;
(iii) waiting for a time period necessary for the photosensitizing agent to achieve an effective tissue concentration at the desired site; and
(iv) photoactivating the photosensitizing agent.

In a further aspect, provided herein is a method of photodynamic treatment or diagnosis of cancer or a non-cancerous condition in a patient, said method comprising:
(i) evacuating the lower part of the gastrointestinal system of the patient;
(ii) administering to the patient a hyperosmotic preparation as herein described;
(iii) insufflating the lower part of the gastrointestinal system, e.g. with air or a gas; and
(iv) photoactivating the photosensitizing agent.

In a further aspect, provided herein is a method of photodynamic treatment or diagnosis of cancer or a non-cancerous condition in a patient, said method comprising:
(i) evacuating the lower part of the gastrointestinal system of the patient;
(ii) insufflating the lower part of the gastrointestinal system, e.g. with air or a gas;
(iii) administering to the patient a hyperosmotic preparation as herein described;
(iv) waiting for a time period necessary for the photosensitizing agent to achieve an effective tissue concentration at the desired site;
(v) insufflating the lower part of the gastrointestinal system, e.g. with air or a gas; and
(vi) photoactivating the photosensitizing agent.

In one embodiment, in the method for using the hyperosmotic preparations, prior to step (i) the lower part of the gastrointestinal system of the patient is evacuated, preferably by using a cleansing enema or a laxative.

Following administration of the enema preparation, a balloon may be inserted into the opening of the rectum to avoid leakage of the product. To enhance homogenous filling of the whole colon the patient may be moved from one side to the other.

The preparations described herein may additionally comprise, or be administered in combination with, an anti-cancer agent. Also provided herein are products which comprise a hyperosmotic preparation as herein described and at least one anti-cancer agent, and their use in treating cancer. Further provided are kits or packs containing a hyperosmotic preparation as herein described, and separately an anti-cancer agent for simultaneous, separate or sequential use in a method of treating cancer.

Exemplary anti-cancer agents include anti-neoplastic agents. Representative examples of anti-neoplastic agents include alkaloids, e.g. vincristine, vinblastine, vinorelbine, topotecan, teniposiode, paclitaxel, etoposide and docetaxel, alkylating agents, e.g. alkyl sulfonates such as busulfan, aziridines, e.g. carboquone, ethylenimines and methylmelamines, nitrogen mustards, e.g. chlorambucil, cyclophosphamide, estramustin, ifosfamide and melphalan, nitrosurea derivatives, e.g. carmustine and lomustine, antibiotics, e.g. mitomycins, doxorubicin, daunorubicin, epirubicin and bleomycins, antimetabolites, e.g. folic acid analogues and antagonists such as methotrexate and raltitrexed, purine analogues, e.g. 6-mercaptopurine, pyrimidine analogues, e.g. tegafur, gemcitabine, fluorouracil and cytarabine, cytokines, enzymes such as L-asparginase, ranpirnase, immunomodulators, e.g. interferons, immunotoxins, monoclonal antibodies, taxanes, topoisomerase inhibitors, platinum complexes like carboplatin, oxaliplatin and cisplatin and hormonal agents such as androgens, estrogens, antiestrogens and aromatase inhibitors. Other anti-neoplastic agents may include, for example, imiquimod, irenotecan, leucovorin, levamisole, etoposide and hydroxyurea.

Preferred anti-cancer agents include, for example, 5-fluorouracil, imiquimod, cytokines, mitomycin C, epirubicin, irenotecan, oxalipatin, leucovorin, levamisole, doxorubicin, cisplatin, etoposide, doxirubicin, methotrexate, taxanes, topoisomerase inhibitors, hydroroxyurea and vinorelbine. Yet more preferred for use as anti-cancer agents are antibiotics such as mitomycin and pyrimidine analogues such as 5-fluorouracil.

The preparations disclosed herein may additionally comprise, or be administered in combination with, one or more non-photosensitizing agents. Products which comprise a hyperosmotic preparation as herein described and at least one non-photosensitizing agent, and their use in treating cancer or a non-cancerous condition are therefore also provided herein. Such agents may, for example, include antibiotics for treatment of various bacterial infections, anti-inflammatory agents like 5-aminosalicylic acid and derivatives thereof for the treatment of inflammatory bowel diseases and inflammatory conditions in the lower gastrointestinal tract, or other drugs such as 5-HT ligands and steroids. Provided herein are such preparations and their use in medicine (e.g. in treating a non-cancerous condition). Further provided herein are kits or packs containing a hyperosmotic preparation as herein described, and separately a non-photosensitizing agent, for simultaneous, separate or sequential use in a method of medical treatment (e.g. a method of treating a non-cancerous condition).

In the case of anti-inflammatory agents, such agents may also be used orally in a period before any enema procedure and/or may be present in the products which are used to evacuate the lower part of the gastrointestinal system prior to the instillation of the enema preparation. Hence the use of oral anti-inflammatory agents and/or laxatives or cleansing enemas comprising anti-inflammatory agents is preferably followed by instillation of an enema preparation as herein described. The use of anti-inflammatory agents may be beneficial to help to reduce unspecific fluorescence of inflammatory lesions which may lead to “false-positive” results in the PDD procedure.

Diagnostic agents may also be present in the preparations herein described or, alternatively, may be administered in combination with the hyperosmotic preparations. Also provided herein is a hyperosmotic preparation as herein described and a diagnostic agent, for example an X-ray contrast agent or an MRI contrast agent. A kit or pack containing a hyperosmotic preparation as hereinbefore defined, and separately a diagnostic agent for instance an X-ray contrast agent or an MRI contrast agent, for simultaneous, separate or sequential use in a method of diagnosis or as a follow-up to treatment of cancer or a non-cancerous condition, is also provided herein.

The preferred X-ray contrast agents to be used according to the procedures disclosed herein are barium sulphate and non-ionic X-ray contrast agents like for example iohexyl, iopamoidol and iodixanol. The formulations comprising an X-ray contrast agent may comprise, for example, 2-30 weight % of the X-ray contrast agent in addition to the photosensitizing agent. Suitable MRI contrast agents are those based on iron, manganese or gadolinium like gadopentetate. When used in combination with an X-ray contrast agent or an MRI contrast agent, the hyperosmotic preparations herein described are able to provide double contrast enhancement, i.e. PDD plus X-ray or PDD plus MRI. Alternatively, the contrast agent might be present in the formulation to visually check in X-ray imaging or MRI that the formulation is present in the whole colon or at least present at the site or area of interest.

The preparations herein described may be administered in combination with a second photosensitizing agent, preferably one comprising 5-ALA or a precursor or derivative thereof. The second agent may be administered by an alternative mode of administration, e.g. orally.

Also provided herein is a kit or pack containing a hyperosmotic preparation as herein described, and separately an oral composition comprising a second photosensitizer which comprises 5-ALA or a precursor or derivative thereof. The oral composition is preferably an oral composition intended for PDD or PDT of the lower part of the gastrointestinal system. Such compositions may be solid formulations like tablets, pellets, capsules containing non-aqueous formulations. Suitable formulations include those described in WO 2009/074811.

The hyperosmotic preparations herein described may be provided in “ready-to-use” form. Alternatively, these may be provided in a kit or pack comprising one or more separate components, e.g. two components which when mixed together provide the desired preparation. Also provided herein are hyperosmotic preparations comprising two components that are mixed before use. This two-component may comprise two vials; one vial contains a preparation comprising 5-ALA or a precursor or derivative thereof which preferably will be formulated as a solid, optionally with other solid materials; and the second vial contains a hyperosmotic liquid. The solid material from the first vial is dissolved or dispersed in the liquid from the second vial immediately prior to use at the hospital or clinic.

Alternatively, the hyperosmotic preparations herein described may be comprised in a three component kit or pack comprising three vials; one vial contains a preparation comprising 5-ALA or a precursor or derivative thereof which preferably will be formulated as a solid, optionally with other solid materials; the second vial contains the hyperosmotic agents as described herein and the third vial contains a liquid, preferably an aqueous liquid, e.g. water. The content of the first and second vials are dissolved or dispersed in the liquid from the third vial, preferably immediately before use.

“Ready-to-use” preparations will generally be provided in a “single-use” sealed disposable container of plastic or glass. Those formed of a polymeric material should have sufficient flexibility for ease of use by an unassisted patient. Plastic containers can be made of polyethylene. These containers may comprise a tip for direct introduction into the rectum. Such containers may also comprise a tube between the container and the tip. The tip is preferably provided with a protective shield which is removed before use. Optionally the tip has a lubricant to improve patient compliance.

Prior to administration of the hyperosmotic preparation it is usual to first cleanse the colonic area. This may be achieved using an enema intended for cleansing purposes. Also provided herein is a kit or pack containing a hyperosmotic enema preparation as hereinbefore defined, and separately a second enema for cleansing. This second enema may be any commercially available cleansing enema, such as those herein described.

Any of the kits or packs herein described may further optionally comprise a balloon intended for use in preventing leakage of the enema, especially that containing the photosensitizing agent, following administration. Such kits or packs may further include instructions for use of the product or products in a method of photodynamic therapy or diagnosis as herein described.

The enema preparation can be administered by known intra-colonic methods. For example, when provided in a flexible container this can be administered to a patient by squeezing the container; this can be done by the patient or by a nurse or other medical assistant. Another option is to administer the enema based on gravity forces by placing the enema above the patient or the enema might be administered using various apparatus available in the clinic or at the doctor's office. Such apparatus are for example described in U.S. Pat. No. 4,504,270, U.S. Pat. No. 4,419,099 and U.S. Pat. No. 4,117,847. The amount of the enema preparation administered will be selected according to its use, the age, sex and other conditions of the patient, and the severity of the condition. The total volume of the enema may vary, for example, from 30 ml to 1500 ml. An enema volume for diagnosis or therapy of, for example, colorectal cancer may be around 500 ml.

After administration of the hyperosmotic preparation containing the photosensitizer, the site to be treated or diagnosed is exposed to light to achieve the desired photosensitizing effect. The length of time following administration at which the light exposure takes place will depend on the nature of the enema, e.g. whether this is in liquid or foam form, whether this contains any delayed release agents, etc., the condition to be treated or diagnosed, etc. Generally, it is necessary that the photosensitizer should reach an effective tissue concentration at the site of the condition (e.g. cancer) prior to photoactivation. This can generally take in the region of from 0.5 to 24 hours, preferably 0.5 to 3 hours.

In a preferred treatment or diagnosis procedure, the photosensitizer is applied to the affected site followed by irradiation e.g. after a period of about 0.5 to 3 hours. If necessary, e.g. during treatment, this procedure may be repeated, e.g. up to a further 3 times, at intervals of up to 30 days, e.g. 7-30 days. In those cases where this procedure does not lead to a satisfactory reduction in, or complete healing of, the condition (e.g. cancer), an additional treatment may be performed several months later.

For therapeutic purposes, methods for irradiation of different areas of the body, e.g. by lamps or lasers are well known in the art (see for example Van den Bergh, Chemistry in Britain, May 1986 p. 430-439). The wavelength of light used for irradiation may be selected to achieve an efficacious photosensitizing effect. The most effective light is light in the wavelength range of from about 300 to about 800 nm, for example from about 400 to about 700 nm where the penetration of the light is found to be relatively deep. The irradiation will in general be applied at a dose level of 10 to 100 Joules/cm² with an intensity of 20-200 mW/cm² when a laser is used or a dose of 10-100 J/cm² with an intensity of 50-150 mW/cm² when a lamp is applied. For treatment, irradiation is preferably performed for 5 to 30 minutes, preferably for 15 minutes. For diagnosis, irradiation is preferably performed during the whole diagnostic procedure or during a part thereof, e.g., when combined with white light detection. A single irradiation may be used or alternatively a light split dose in which the light dose is delivered in a number of fractions, e.g. a few minutes to a few hours between irradiations, may be used. Multiple irradiations may also be applied. Devices specifically adapted for use in irradiating the colonic area will preferably be used, e.g. an endoscope.

For diagnostic use, the area is preferably first inspected using white light. Suspicious areas are then exposed to blue light (for example, ranging from about 400 to about 450 nm). The emitted fluorescence (635 nm) is then used to selectively detect affected cancerous or non-cancerous tissues having a higher metabolic activity than healthy tissue. When carrying out diagnosis, it is preferable to use blue light using a device e.g. an endoscope and assessing the fluorescence.

The products and methods herein disclosed may be used to treat and/or diagnose cancer or non-cancerous conditions in the lower gastrointestinal tract, in particular in the large intestine (colon), especially in the sigmoid colon, the descending colon and the rectum. Such conditions include inflammatory bowel diseases, colorectal cancer, ulcerative colitis, Crohn's disease, irritable bowel disease, etc. Inflammatory bowel diseases are inflammatory diseases of the large and small intestines which may be caused by a number of factors. In most patients the regions affected extend over a wide range of the colon, e.g. to the descending colon or transverse colon. Use of the preparations herein described ensures that the desired therapeutic or diagnostic effects are achieved because the active ingredients can directly reach the affected regions.

The invention will now be described in more detail by way of the following non-limiting examples and with reference to the accompanying figures in which:

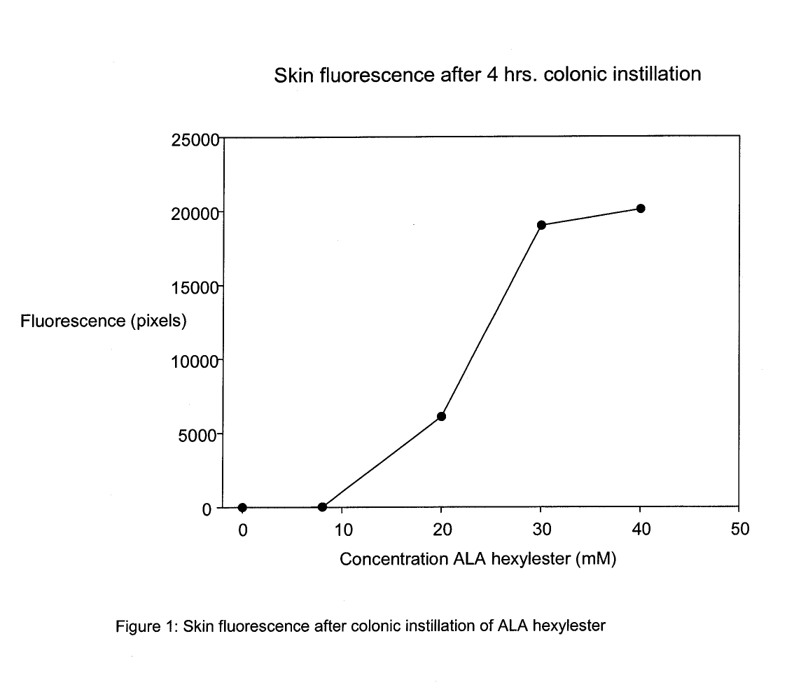

FIG. 1—shows the skin fluorescence after colonic instillation of ALA hexylester in mice in accordance with Example 5; and

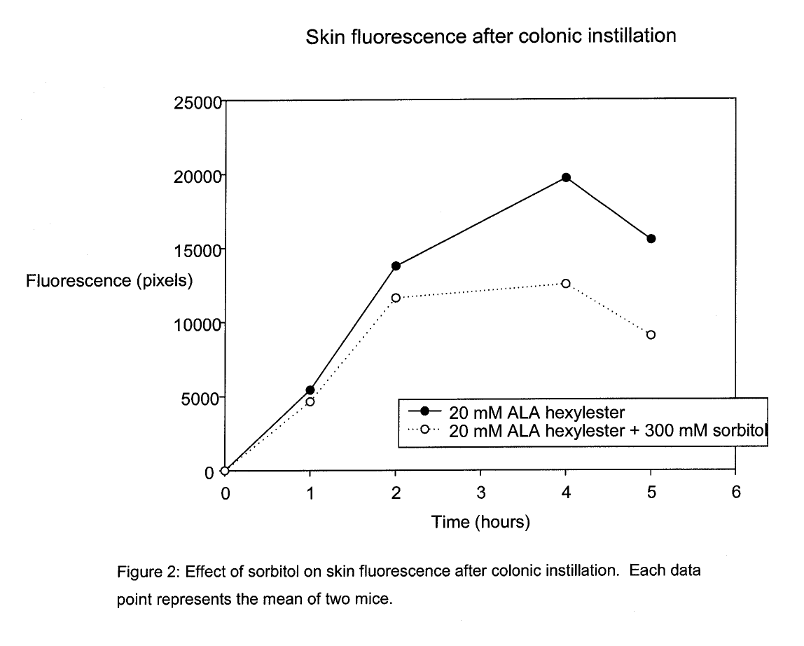

FIG. 2—shows the effect of sorbitol on skin fluorescence after colonic instillation of ALA hexylester in mice in accordance with Example 6.

### EXAMPLE 1

### Powder for Preparation of an Enema Comprising 5-ALA Hexyl Ester Hydrochloride and Sorbitol

| | |
|---|---|
| Sorbitol | 27.32 g |
| 5-ALA hexyl ester hydrochloride | 0.315 g |

5-ALA hexyl ester hydrochloride and sorbitol are mixed using a powder mixer. The resulting powder (27.635 g) is filled into a 600 ml plastic flask. Prior to use, 500 ml water is added and the mixture is shaken for 2 minutes before the solution is administered as an enema. The solution comprises 300 mmol sorbitol per liter and 2.5 mmol 5-ALA hexyl ester per liter (osmolarity: 305 mOsm/l).

### EXAMPLE 2

### Powder for Preparation of an Enema Comprising 5-ALA Hexyl Ester Hydrochloride and Mannitol

| | |
|---|---|
| Mannitol | 54.65 g |
| 5-ALA hexyl ester hydrochloride | 0.252 g |

5-ALA hexyl ester hydrochloride and mannitol are mixed using a powder mixer. The resulting powder (54.902 g) is filled into a 600 ml plastic flask. Prior to use, 500 ml water is added and the mixture is shaken for 2 minutes before the solution is administered as an enema. The solution comprises 600 mmol mannitol per liter and 2 mmol 5-ALA hexyl ester per liter (osmolarity: 604 mOsm/l).

### EXAMPLE 3

### Powder for Preparation of an Enema Comprising 5-ALA Hexyl Ester Hydrochloride and Polyethylene Glycol

| | |
|---|---|
| Macrogol 3350* | 75.00 g |
| 5-ALA hexyl ester hydrochloride | 25.18 g |

5-ALA hexyl ester hydrochloride and Macrogol 3350 are mixed using a powder mixer. The resulting powder (100.18 g) is filled into a 1200 ml plastic flask. Prior to use, 1000 ml physiological saline (0.15 M NaCl) is added and the mixture is shaken for 2 minutes before the solution is administered as an enema. The solution comprises 100 mmol 5-ALA hexyl ester per liter and 22 mmol Macrogol 3350 per liter (osmolarity: 326 mOsm/l).
Note that this product contains 13.125 g PEG 3350 and electrolytes to be dissolved in 125 ml of water which gives 105 mg/ml (31 mM) PEG 3350. This concentration of PEG 3350 in a balanced electrolyte solution corresponds to 256 mOsmol/l (Böhmer et al. Eur. J. Geriatrics 10 (1): 33-40, 2008). Further, the solution contains 48 mM NaCl, 17 mM NaHCO₃ and 5 mM KCl. The electrolytes will all dissociate in solution (the NaHCO₃ is expected to dissociate into Na⁺ and HCO₃⁻). The osmolarity of the electrolytes will therefore correspond to (48×2+17×2+5×2) mOsmol/l=140 mOsmol/l. Total osmolarity of the product (PEG+electrolytes) therefore equals 396 mOsmol/l.

### EXAMPLE 4

### Powder and Solution for Preparation of an Enema Comprising 5-ALA Hexyl Ester Hydrochloride and Sorbitol

| | | |
|---|---|---|
| Sorbitol | 27.32 | g |
| 5-ALA hexyl ester hydrochloride | 0.63 | g |
| Carboxymethyl cellulose | 0.5 | g |
| Paraben mixture | 50 | mg |

| | |
|---|---|
| Water | q.s |

Carboxymethyl cellulose is added to water during stirring at 60° C. The aqueous mixture is cooled and the paraben mixture is added. The aqueous mixture is filled into a 600 ml plastic container (osmolarity: 310 mOsm/l).

5-ALA hexyl ester hydrochloride and the other components are mixed using a powder mixer. The resulting powder (28.50 g) is added to the pre-heated aqueous mixture at 37° C. prior to use. The mixture is shaken thoroughly for 5 minutes and administered as an enema. The total volume of the enema is 500 ml, and the solution comprises 300 mmol sorbitol per liter and 5 mmol 5-ALA hexyl ester per liter.

### EXAMPLE 5

### Study: Systemic Uptake from the Colon

Animals

Experiments were performed in hairless mice weighing 20 g (C3.Cg/TifBomTac-hr). The animals were kept in special cages (12090D Eurostandard Type III cage with a raised bottom grid 1290D-150 from Scanbur) to ensure that they did not eat their own faeces during fasting. The animals were fasted for 24 hours but with free access to water before the experiment. Prior to administration of the test preparation, the mice were anaesthetised with Hypnorm/Dormicum. This was also repeated every second hour.

Assessment of Systemic Uptake

When ALA hexylester is administered sytemically, it induces PpIX formation in the entire body. The systemic uptake of ALA hexylester after colonic administration was therefore assessed as skin fluorescence. To assess the PpIX skin fluorescence, the mice were photographed using a fluorescence camera (Medeikonos PDD/PDT, Medeikonos AB, Gothenburg, Sweden) at excitation wavelengths of 365 and 405 nm and 2 sec illumination time. Each photo was calibrated to a fluorescence standard (Uranyl Standard, J&M, Analytische Mess and Regeltecknik, GmbH, Hamburg, Germany) and adjusted for background fluorescence. The mean amount of PpIX fluorescence in each image was calculated by means of image analyzer-software (MatLab 7.2.0.232, Math-Works, Natick, Mass., USA).

Colonic Administration

All dilutions were performed in 0.9% NaCl (150 mM=300 mOsmol/l). For the colon instillation, a 1000 μl micropipette with a plastic tip was used. Preliminary experiments showed that it was possible to instill the desired volume (0.6 ml) without perforating the colon. In all experiments, instillation lasted until the experiment was finished (approx. 5 hours).

Results from Colonic Administration

The purpose was to find a suitable concentration of ALA hexylester that resulted in a significant skin fluorescence without causing any apparent toxicity to the animals. Increasing doses (8 to 40 mM ALA hexylester) were given by anal administration to mice and the resulting skin fluorescence measured as described. The results are shown in FIG. 1. This shows a clear dose-response relationship between the concentration of ALA hexylester in the enema and the resulting skin fluorescence. The resulting systemic fraction was proportional to the concentration of ALA hexylester in the enema.

It can be seen from FIG. 1 that the concentration of ALA hexylester that has been used clinically (8 mM) gave no skin fluorescence, whereas concentrations≧30 mM appeared to be saturating.

### EXAMPLE 6

### Study: Hypertonic Conditions in the Colon

For this experiment, 20 mM ALA hexyl ester was chosen for colonic administration since this was well below the saturation levels but gave reasonable levels of skin sensitivity (see Example 5). The ALA hexylester was dissolved as the corresponding hydrochloride salt. 20 mM ALA hexylester constitutes an osmolarity of approx. 40 mOsmol/l.

The purpose of this study was to investigate the effect of adding sorbitol to an enema formulation containing 20 mM ALA hexylester in an attempt to reduce the systemic uptake of the photosensitizer. It was discovered in pilot experiments that sorbitol at concentrations above 300 mM gave diarrhea. Therefore, the effect of 300 mM sorbitol in 0.9% NaCl (600 mOsmol/l) was investigated by comparing the skin fluorescence in two mice that were given 20 mM ALA hexylester (in 0.9% NaCl) with the skin fluorescence in two mice that had received 20 mM ALA hexylester and 300 mM sorbitol. The osmolarities of these solutions are outlined below.
1. 20 mM ALA hexylester in 0.9% NaCl: (40+300) mOsmol/l=340 mOsmol/l
2. 20 mM ALA hexylester and 300 mM sorbitol in 0.9% NaCl: (40+300+300) mOsmol/l=640 mOsmol/l (hypertonic preparation)

The results are shown in FIG. 2. This shows a clear reduction in skin fluorescence indicating a significant reduction in the systemic fraction of ALA hexylester.

1. A hyperosmotic preparation comprising a photosensitizing agent and at least one hyperosmotic agent, wherein the photosensitizing agent comprises 5-ALA, a precursor or a derivative thereof.

2. The hyperosmotic preparation of claim 1 which comprises at least one hyperosmotic agent selected from the group consisting of salts, sugars, sugar alcohols, glycerol, polyols and combinations thereof.

3. The hyperosmotic preparation of claim 2, wherein the hyperosmotic agent comprises magnesium sulphate, magnesium hydroxide, magnesium citrate, magnesium chloride, sodium phosphate, or any combination thereof.

4. The hyperosmotic preparation of claim 2, wherein the hyperosmotic agent comprises sorbitol, mannitol, lactitol, xylitol, lactulose, fructose, galactose, lactose, or any combination thereof.

5. The hyperosmotic preparation of claim 2, wherein the hyperosmotic agent comprises a polyether polyol, preferably a polyethylene glycol (PEG) or polyethylenepolypropylene glycol (PPG).

6. The hyperosmotic preparation of claim 1, wherein the photosensitizing agent comprises a 5-ALA derivative or a pharmaceutically acceptable salt thereof.

7. The hyperosmotic preparation of claim 1, wherein the photosensitizing agent is a compound of formula I or a pharmaceutically acceptable salt thereof:
R²₂N—CH₂COCH₂—CH₂CO—OR¹ (I)
wherein
R¹ represents a substituted or unsubstituted alkyl group; and
R² each independently represents a hydrogen atom or a group R¹.

8. The hyperosmotic preparation of claim 7, wherein each R² represents hydrogen and R¹ represents an unsubstituted alkyl group.

9. (canceled)

10. The hyperosmotic preparation of claim 1, wherein said preparation is in the form of an enema.

11. A method of photodynamic treatment or diagnosis of cancer or a non-cancerous condition in the lower part of the gastrointestinal tract comprising administration of the hyperosmotic preparation of claim 1.

12. The method of claim 11, wherein the cancer or non-cancerous condition in the lower part of the gastrointestinal tract is a non-cancerous condition selected from inflammatory bowel disease, ulcerative colitis, Crohn's disease and irritable bowel syndrome.

13. A method of photodynamic treatment or diagnosis of cancer or a non-cancerous condition in the lower part of the gastrointestinal tract, wherein said method comprises the steps of:
(i) administering to a patient an effective amount of the hyperosmotic preparation of claim 1;
(ii) optionally waiting for a time period for the photosensitizer to achieve an effective tissue concentration at the desired site; and
(iii) photoactivating the photosensitizer.

14. The method of claim 13, wherein prior to step (i) the lower part of the gastrointestinal system of said patient is evacuated.

15. The hyperosmotic preparation of claim 1, wherein the hyperosmotic preparation comprises at least one of the following:
a salt, sugar, sugar alcohol, glycerol, polyol, or combination thereof;
magnesium sulphate, magnesium hydroxide, magnesium citrate, magnesium chloride, sodium phosphate, or combination thereof;
sorbitol, mannitol, lactitol, xylitol, lactulose, fructose, galactose, lactose, or combination thereof; and
a polyether polyol;
and further wherein the photosensitizing agent comprises a 5-ALA ester or a pharmaceutically acceptable salt thereof.

16. The hyperosmotic preparation of claim 15, wherein the photosensitizing agent is a compound of formula I or a pharmaceutically acceptable salt thereof:
R²₂N—CH₂COCH₂—CH₂CO—OR¹ (I)
wherein
R¹ represents a substituted or unsubstituted alkyl group; and
R² each independently represents a hydrogen atom or a group R¹.

17. The hyperosmotic preparation of claim 16, wherein each R² represents hydrogen and R¹ represents an unsubstituted alkyl group.

18. The hyperosmotic preparation of claim 17, wherein each R¹ represents an unsubstituted C₁-C₆ alkyl group.

19. The hyperosmotic preparation of claim 18, wherein said preparation is in the form of an enema.

20. The method of claim 11, wherein the hyperosmotic preparation is administered to the colon and/or rectum.

21. The method of claim 11, wherein the hyperosmotic preparation is the hyperosmotic preparation of claim 18.
